(19) United States
(12) Patent Application Publication
(10) Pub. No.: US 2023/0256116 A1
(43) Pub. Date: Aug. 17, 2023

(54) NANOCOMPOSITES OF NITROGEN-DOPED GRAPHENE OXIDE AND MANGANESE OXIDE FOR PHOTODYNAMIC THERAPY AND MAGNETIC RESONANCE IMAGING

(71) Applicant: King Saud University, Riyadh (SA)

(72) Inventors: Haseeb Ahmad Khan, Riyadh (SA); Sara Tariq Alrashood, Riyadh (SA); Mohammed Rafi Shaik, Riyadh (SA); Aishah Ali Ekhzaimy, Riyadh (SA)

(73) Assignee: King Saud University, Riyadh (SA)

(21) Appl. No.: 18/308,537

(22) Filed: Apr. 27, 2023

(51) Int. Cl. A61K 47/69; A61K 47/52; A61K 49/08

(52) U.S. Cl. CPC A61K 47/6929; A61K 47/52; A61K 49/085

(57) ABSTRACT

The present invention relates to a NDG-Mn₃O₄ nanocomposite comprising a nitrogen doped graphene (NDG) and Mn₃O₄ nanoparticles. The NDG-Mn₃O₄ nanocomposite is useful in bimodal performance including photodynamic therapy (PDT) and magnetic resonance imaging (MRI). The NDG-Mn₃O₄ nanocomposites of the present invention caused significant cell death under laser irradiation, while control and Mn₃O₄ nanoparticles showed negligible cell death.

### FIELD OF THE INVENTION

The present disclosure generally relates to nanocomposites. Specifically, the present disclosure relates to a nitrogen doped graphene (NDG)-Mn₃O₄ nanocomposite comprising a nitrogen doped graphene (NDG) and Mn₃O₄ nanoparticles. The NDG-Mn₃O₄ nanocomposite is useful in bimodal performance including photodynamic therapy (PDT) and magnetic resonance imaging (MRI). The NDG-Mn₃O₄ nanocomposites of the present invention cause significant cell death under laser irradiation.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Cancer is one of the deadliest and costliest diseases and is the second leading cause of death worldwide. Photodynamic therapy (PDT) is a promising treatment modality for cancer with minimal side effects and is expected to replace traditional chemotherapy, which is associated with numerous adverse effects. PDT involves combination of light and a photosensitizer (PS), which is activated by absorption of light of a specific wavelength, causing the generation of potentially toxic reactive oxygen species (ROS) that induce a cascade of intracellular molecular events resulting in targeted tissue damage [Avci, P.; Erdem, S.S.; Hamblin, M.R. J. Biomed. Nanotechnol. 2014, 10, 1937-1952]. Sun et al. have reviewed the application of metal-based nanoparticles (NPs) for PDT of cancer [Sun J et al. Molecules, 2018, 23, 1704]. Metal oxide-based nanomaterials have also significantly impacted the landscape of healthcare, including in the areas of diagnosis and therapeutic applications. Metal oxides have demonstrated great potential in PDT and magnetic resonance imaging (MRI) in diagnostic radiology. A majority of the transition metals-based oxides offer several advantages in the field of biomedicines due to their biocompatibility and non-toxicity. Even some of them, such as iron oxide, have been approved as an MRI contrast agent by concerned authorities [Wang, D. et al, ACS Nano 2014, 8, 6620-6632]. Therefore, metal oxide NPs have the potential to serve as both therapeutic and imaging agents, particularly, manganese oxide (Mn₃O₄) nanoparticles are considered as effective in tumor diagnosis and treatment due to their decent biocompatibility, in-vivo imaging performance and tumor microenvironment (TME) responsiveness. Notably, Mn₃O₄ is consists of Mn²⁺ and Mn³⁺, due to which it is extremely sensitive to the redox environment in the cell and rapidly decomposes upon exposure to glutathione (GSH). Tumor specific antibodies functionalized Mn₃O₄ NPs were applied as T1 MRI contrast agent for selective imaging of cancer cells [Na, H.B. et al, Angew. Chem. 2007, 46, 5397-5401]. To avoid some limitations, such as aggregation, poor water dispersibility, high dermal toxicity and low clearance of these NPs, several stabilizing ligands have applied to the surfaces of NPs which make them stable and suitable for therapeutic applications. For instance, folic acid (FA) has been used as ligand for targeting folate receptors (FR), a tumor-associated protein over-expressed in cancer cells having high binding affinity toward folic acid. Recently, transitional metal oxide NPs including Mn₃O₄, have been effectively combined with a variety of 2D materials, specially graphene, which has received promising attention for phototherapy due to its excellent photosensitizer properties.

Graphene is made up of a single layer of carbon atoms arranged in a honeycomb structure, demonstrating specific combination of physiochemical properties, such as, high surface area (2630 m²g⁻¹), optimal thermal conductivity (~5000 Wm K⁻¹), and remarkable optical transparency, which make it excellent candidate for drug delivery and therapeutic applications. However, its hydrophobicity causes irreversible agglomeration, which is a great obstacle for utilizing its drug career properties. On the other hand, the oxidation of graphene into graphene oxide (GO) significantly reduces its aggregation tendency. GO exhibits amphiphilic nature due to the presence of hydrophobic graphene moiety and hydrophilic edges; the former property is important for carrying water-insoluble drugs through non-covalent bonding, π-π stacking or hydrophobic interaction or hydrogen bonding whereas the latter property not only provides anchor sites for functionalization but also maintains colloidal stability due to negative surface charge. When dispersed in water, GO attains a negative surface charge due to ionization of hydroxyl and carboxylic groups. The magnitude of this negative charge is sufficient to cause electrostatic repulsion resulting in stable dispersion of GO in water [Li, D et al, Nat. Nanotechnol. 2008, 3, 101-105].

The water dispersibility of GO is considered better than the water dispersibility of carbon nanotubes (CNTs). However, GO contains a variety of oxygen containing random functional groups, which inhibit the homogeneous binding of the NPs on its surface. Biological investigations of GO, both in-vitro and in-vivo have no consensus results and sometimes the results are in contradiction [Kiew, S.F. et al, J. Control. Release, 2016, 226, 217-228].

There is therefore a need in the art to develop nanocomposite, which is useful in photodynamic therapy as well for magnetic resonance imaging (MRI). The present invention satisfies the existing needs, as well as others, and generally overcomes the side effects of the chemotherapy.

### OBJECTS OF THE INVENTION

Primary objective of the present disclosure is to provide a novel nanocomposite for photodynamic therapy and magnetic resonance imaging.

Another objective of the present disclosure is to provide a nanocomposite comprising nitrogen doped graphene oxide (NGO) and Mn₃O₄ nanoparticles.

Another objective of the present disclosure is to provide a method of preparation of nanocomposite comprising nitrogen doped graphene oxide (NGO) and Mn₃O₄ nanoparticles.

Another objective of the present disclosure is to provide a method of treating tumors or imaging targeted tissue by photodynamic therapy using nanocomposite comprising nitrogen doped graphene oxide (NGO) and Mn₃O₄ nanoparticles.

### SUMMARY OF THE INVENTION

The present disclosure relates to a NDG-Mn₃O₄ nanocomposite comprising a nitrogen doped graphene (NDG) and Mn₃O₄ nanoparticles. The NDG-Mn₃O₄ nanocomposite is useful in bimodal performance including photodynamic therapy (PDT) and magnetic resonance imaging (MRI). The NDG-Mn₃O₄ nanocomposites of the present invention caused significant cell death under laser irradiation, while control and Mn₃O₄ nanoparticles showed negligible cell death.

In an aspect, the present invention relates to a nanocomposite comprising a nitrogen doped graphene oxide conjugated with Mn₃O₄ nanoparticle.

In another aspect of the present invention, particle size of the nanocomposite is in a range of 5 nm to 15 nm.

In another aspect of the present invention, the nitrogen doped graphene oxide and the Mn₃O₄ nanoparticle is present in the nanocomposite in a ratio of 1:1.

In another aspect of the present invention, the nanocomposite is obtained using milling process.

In yet another aspect, the present invention relates to a process of preparation of a nanocomposite as claimed in claim 1, wherein the process comprises the steps of:
(a) reacting manganese (II) acetylacetonate with oleylamine at a temperature in a range of 150° C. to 170° C. for a period of 8 to 12 hours to obtain Mn₃O₄ nanoparticles;
(b) reacting a suspension of graphene oxide (GO) with hydrazine hydrate in presence of a base at a temperature range of 85° C. to 95° C. for a period of 1 to 4 hours to obtain nitrogen doped graphene oxide;
(c) milling the nitrogen doped graphene oxide and the Mn₃O₄ nanoparticles for a time period in a range of 14 hours to 17 hours to obtain a nanocomposite comprising the nitrogen doped graphene oxide conjugated with the Mn₃O₄ nanoparticle.

In another aspect of the present invention, the manganese (II) acetylacetonate and oleylamine used in the process of preparing nanocomposite is present in a molar ratio of 1:25.

In another aspect of the present invention, the base used in the process of preparing nanocomposite is ammonium hydroxide or potassium hydroxide.

In another aspect of the present invention, the nitrogen doped graphene oxide and the Mn₃O₄ nanoparticles used in the process of preparation of nanocomposite is present in 1:1 ratio.

In another aspect of the present invention, the temperature in step (a) is 160° C. and in step (b) is 90° C.

In yet another aspect, the present invention relates to a method of treating a cancer or imaging targeted tissue in a subject, comprising:
(a) administering to the subject in need thereof an effective amount of a nanocomposite comprising nitrogen doped graphene oxide and the Mn3O4 nanoparticles; and
(b) exposing cancer cell or tissue of the subject to laser irradiation in presence of fluorescein diacetate (FDA) and propidium iodide (PI) for a sufficient amount of time to obtain a desired response.

In another aspect of the present invention, the cancer is lung cancer, breast cancer, prostate cancer, brain cancer, colorectal cancer, pancreatic cancer, ovarian cancer, cervical cancer, liver cancer, head/neck/throat cancer, skin cancer, bladder cancer and a hematologic cancer.

In another aspect of the present invention, the imaging is Magnetic Resonance Imaging (MRI).

The following extensive discussion of preferred embodiments will reveal several objects, features, characteristics, and advantages of the inventive subject matter.

### BRIEF DESCRIPTION OF THE FIGURES

FIG. 1: High-resolution transmission electron microscopy (HRTEM) images of the NDG-Mn₃O₄ nanocomposite (a) low magnification image, (b) magnified image, (c) energy-dispersive X-ray spectroscopy of NDG-Mn₃O₄ nanocomposite and (d) particle size distribution of NDG-Mn3O4 nanocomposite.

FIG. 2: XRD pattern of (a) Mn₃O₄ NPs, (b) NDG and (c) NDG-Mn₃O₄ nanocomposite.

FIG. 3: FT-IR spectra of (a) Mn₃O₄ NPs, (b) NDG and (c) NDG-Mn₃O₄ nanocomposite.

FIG. 4: Cytotoxicity analysis showing cell viability of A549 cells treated with different concentrations of Mn₃O₄ and NDG—Mn₃O₄ nanocomposites. Values are means of three replicates ± standard error.

FIG. 5: Cell viability of A549 cells incubated with PBS (control), Triton X-100 (negative control) and different concentrations of, Mn₃O₄ and NDG-Mn₃O₄ nanocomposites in presence of 670 nm laser irradiation (0.1 W/cm²) for 5 min. Data is represented as mean values of three replicates (±) standard deviations. *p< 0.05, **p< 0.01 and ***p< 0.001 versus respective control groups.

FIG. 6: Fluorescence microscopy of A549 cells stained with fluorescein diacetate (green emission for live cells) and propidium iodide (red emission for dead cells) with PBS (control), Mn₃O₄ and NDG—Mn₃O₄ nanocomposites with/without laser irradiation (670 nm, 0.1 W/cm²) for 5 min.

FIG. 7: Effect of SNWE treatment on apoptotic markers in the MCF-7 and MDA-MB-231 cells. The protein expressions was analysed by immunofluorescence method, the A) BAX, B) Caspase3, and C) p53, expression were increased in the SNWE treatment which shows the induction of caspase dependent apoptosis in breast cancer cells. The cells were immunostained with anti p53, BAX, Caspase3 antibodies and FITC labelled secondary antibodies. DAPI was used as counter stain for nucleus and the images were acquired with fluorescence microscope.

FIG. 8: T₁-weighted MR imaging of NDG-Mn₃O₄ nanoparticles in aqueous suspension and the T1 relaxivity plot of aqueous suspension of NDG-Mn₃O₄ nanoparticles. The concentration range of 0.06-1.0 mM of Mn is equivalent to approximately 18-152 µg/mL of NDG-Mn₃O₄ nanoparticles.

### DETAILED DESCRIPTION OF THE INVENTION

The embodiments herein and the various features and advantageous details thereof are explained more comprehensively with reference to the non-limiting embodiments that are detailed in the following description. Descriptions of well-known components and processing techniques are omitted so as to not unnecessarily obscure the embodiments herein. The examples used herein are intended merely to facilitate an understanding of the ways in which the embodiments herein may be practiced and to further enable those of skill in the art to practice the embodiments herein. Accordingly, the examples should not be construed as limiting the scope of the embodiments herein.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

As used in the description herein, the meaning of “a,” “an,” and “the” includes plural reference unless the context clearly dictates otherwise. Also, as used in the description herein, the meaning of “in” includes “in” and “on” unless the context clearly dictates otherwise.

As used herein, the terms “comprise”, “comprises”, “comprising”, “include”, “includes”, and “including” are meant to be non- limiting, i.e., other steps and other ingredients which do not affect the end of result can be added. The above terms encompass the terms “consisting of” and “consisting essentially of”.

The present disclosure relates to a NDG-Mn₃O₄ nanocomposite comprising a nitrogen doped graphene (NDG) and Mn₃O₄ nanoparticles. The NDG-Mn₃O₄ nanocomposite is useful in bimodal performance including photodynamic therapy (PDT) and magnetic resonance imaging (MRI). The NDG-Mn₃O₄ nanocomposites of the present invention caused significant cell death under laser irradiation.

In an embodiment, the present invention relates to a nanocomposite comprising a nitrogen doped graphene oxide conjugated with Mn₃O₄ nanoparticle.

In another embodiment of the present invention, particle size of the nanocomposite is in a range of 5 nm to 15 nm. Preferably, the particle size of the nanocomposite is 10±1.7 nm.

In another embodiment of the present invention, the nitrogen doped graphene oxide and the Mn₃O₄ nanoparticle is present in the nanocomposite in a ratio of 1:1.

In another embodiment of the present invention, the nanocomposite is obtained using milling process.

In yet another embodiment, the present invention relates to a process of preparation of a nanocomposite as claimed in claim 1, wherein the process comprises the steps of:
(a) reacting manganese (II) acetylacetonate with oleylamine at a temperature in a range of 150° C. to 170° C. for a period of 8 to 12 hours to obtain Mn₃O₄ nanoparticles;
(b) reacting a suspension of graphene oxide (GO) with hydrazine hydrate in presence of a base at a temperature range of 85° C. to 95° C. for a period of 1 to 4 hours to obtain nitrogen doped graphene oxide;
(c) milling the nitrogen doped graphene oxide and the Mn₃O₄ nanoparticles for a time period in a range of 14 hours to 17 hours to obtain a nanocomposite comprising the nitrogen doped graphene oxide conjugated with the Mn₃O₄ nanoparticle.

In another embodiment of the present invention, the manganese (II) acetylacetonate and oleylamine used in the process of preparing nanocomposite is present in a molar ratio of 1:25.

In another embodiment of the present invention, the base used in the process of preparing nanocomposite is ammonium hydroxide or potassium hydroxide.

In another embodiment of the present invention, the nitrogen doped graphene oxide and the Mn₃O₄ nanoparticles used in the process of preparation of nanocomposite is present in 1:1 ratio.

In another aspect of the present invention, the temperature in step (a) is 160° C. and in step (b) is 90° C.

In yet another embodiment, the present invention relates to a method of treating a cancer or imaging targeted tissue in a subject, comprising:
(a) administering to the subject in need thereof an effective amount of a nanocomposite comprising nitrogen doped graphene oxide and the Mn3O4 nanoparticles; and
(b) exposing cancer cell or tissue of the subject to laser irradiation in presence of fluorescein diacetate (FDA) and propidium iodide (PI) for a sufficient amount of time to obtain a desired response.

In another embodiment of the present invention, the cancer is lung cancer, breast cancer, prostate cancer, brain cancer, colorectal cancer, pancreatic cancer, ovarian cancer, cervical cancer, liver cancer, head/neck/throat cancer, skin cancer, bladder cancer and a hematologic cancer.

In another aspect of the present invention, the imaging is Magnetic Resonance Imaging (MRI).

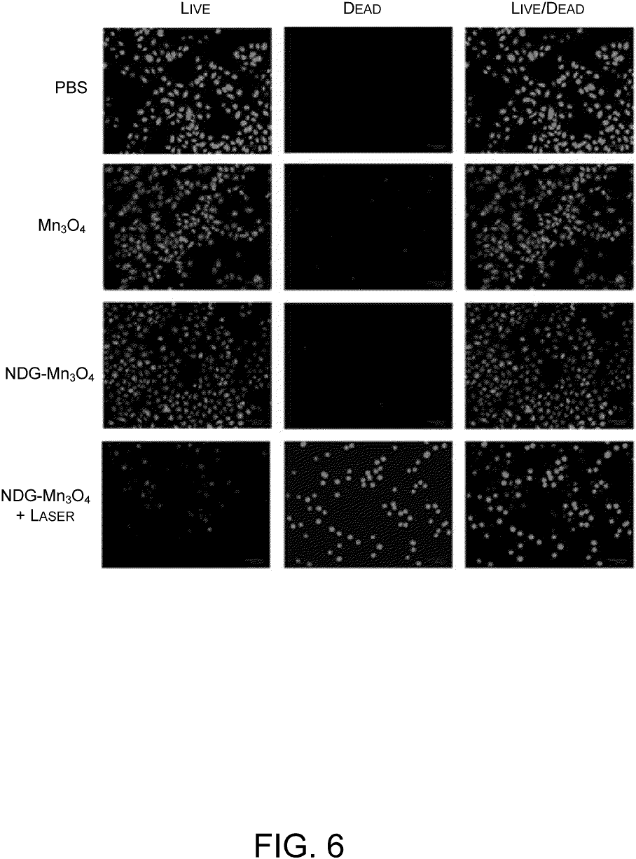

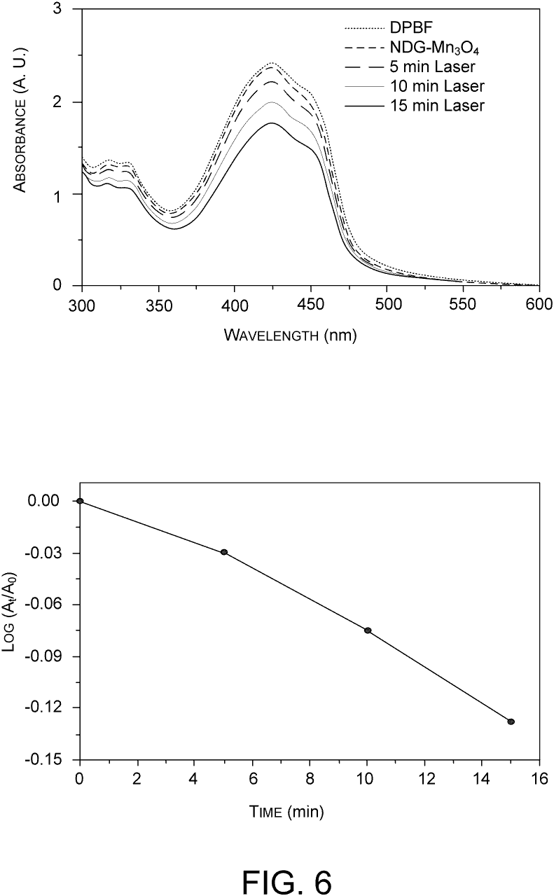

According to the present invention, NDG—Mn₃O₄ nanocomposites did not cause any cytotoxicity unless activated by laser irradiation that resulted in concentration dependent cytotoxicity in lung cancer cells (FIG. 5). These biochemical findings were supported by fluorescence microscopy observations, suggesting that NDG-Mn₃O₄ nanocomposites initiate cytotoxic properties only under laser irradiation (FIG. 6).

In an embodiment of the present invention, the 670 nm laser was used in order to keep the wavelength within the red optical window (620-750 nm).

In another embodiment of the present invention, the NDG-Mn₃O₄ nanocomposites killed 68% of cancer cells which is more effective than GQD-PDA-Mn₃O₄ nanoparticles (51% cell death). The mechanism of laser-induced toxicity of NDG—Mn₃O₄ nanocomposites can be multifactorial. The laser irradiation during PDT triggered the disruption of cellular membranes resulting in a higher cellular uptake of the GQD-PDA-Mn₃O₄ nanoparticles compared to graphene quantum dots. This selective transport across the cell membrane might have been influenced by the size, shape and surface chemistry of nanoparticles.

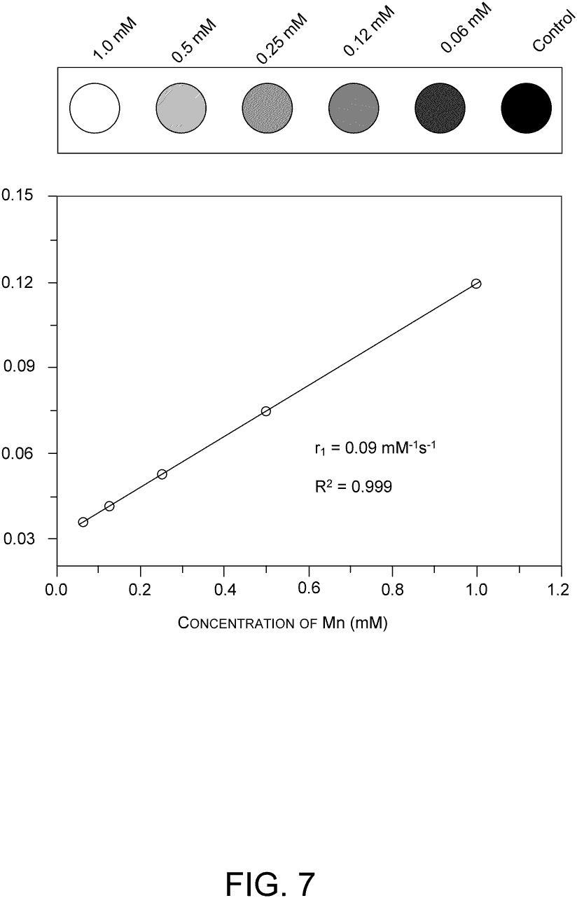

In another embodiment of the present invention, the NDG-Mn₃O₄ nanocomposites caused ¹O₂ generation under laser irradiation in a time-dependent manner and longer exposure to laser irradiation produced excessive ROS generation (FIG. 7).

In another embodiment of the present invention, the results of MRI demonstrated a concentration dependent enhancement of signal intensity with increasing concentration of NDG-Mn₃O₄ nanocomposites (FIG. 8).

The present disclosure may be more fully understood by reference to the following examples:

### EXAMPLES

### Preparation of Mn₃O₄ Nanoparticles

Manganese (II) acetylacetonate was dissolved in oleylamine (molar ratio of manganese (II) acetylacetonate: oleylamine = 1:25) and the mixture was heated at 160 C for 10 h under a nitrogen cover. The resulting product was cooled to room temperature to form a brownish suspension, which was centrifuged at 9000 rpm for 15 min and the supernatant was removed to obtain a brown residue. The precipitate was washed multiple times with ethanol to acquire pure Mn₃O₄ nanoparticles, which were dried under vacuum before use.

### Preparation of Nitrogen-Doped Graphene Oxide (NDG)

Initially, graphite oxide (GO) was synthesized from graphite powder using a modified Hummers method. Briefly, graphite powder (0.5 g) and NaNO₃ (0.5 g) were added to 23 mL of H₂SO₄ and the mixture was stirred for 10 min in an ice bath. Subsequently, KMnO₄ (3 g) was slowly added and after proper mixing, the ice bath was replaced with water bath (35) for 1 h, resulting in the formation of a thick paste. Thereafter, 40 mL of deionized water was added, and the mixture was stirred for 30 min at 90 C. Finally, 100 mL of deionized water was added, followed by the slow addition of 3 mL of H₂O₂. The mixture was allowed to cool, filtered and washed with deionized water. The resulting thick brown paste was dispersed in water and centrifuged at 1000 rpm for 2 min. This step was repeated 4-5 times, until all unsettled particles were removed. The resultant paste was dispersed in water with mild sonication to obtain a suspension of graphene oxide (GO). For nitrogen doping, the resulting suspension was taken in a round bottom flask, to which 4 mL of NH₄OH and 4 mL hydrazine hydrate were added simultaneously. The mixture was stirred for a few minutes, and the flask (equipped with cooling condenser) was put in a water bath controlled at 90° C. for 3 h. The product was collected after been filtered through micropore filters (Whatman filter paper, pore size-20 µm, W&R Balston Limited, Maidstone, Kent, UK), washed by deionized water and freeze-dried.

### Preparation of Nanocomposites of NDG and Mn3O4(NDG—Mn3O4)

Equal amounts of Mn₃O₄ nanoparticles and NDG were milled using a Fritsch Pulverisette P7 planetary ball mill (Idar-Oberstein, Germany). The nanomaterials powder and stainless steel balls (5 mm diameter) with the ball to powder weight ratio of 1:1 were introduced into the stainless steel container. The milling of the powder was performed for 16 h, with intermittent pausing of milling process at regular intervals.

### Characterization of Nanoparticles

The synthesized nanoparticles were characterized for size and physicochemical properties using high resolution transmission electronmicroscopy (JSM-7610F, JEOL, Tokyo, Japan), X-ray diffraction analysis (D2 Phaser X-ray diffractometer, Bruker, Ettlingen, Germany) and FT-IR spectroscopy (Perkin Elmer 1000 FT-IR spectrometer,Waltham, MA, USA). A microplate reader determined the absorbance at 570 nm (Molecular Devices, USA). Percentage cell viability was calculated, and cell-survival curves were constructed.

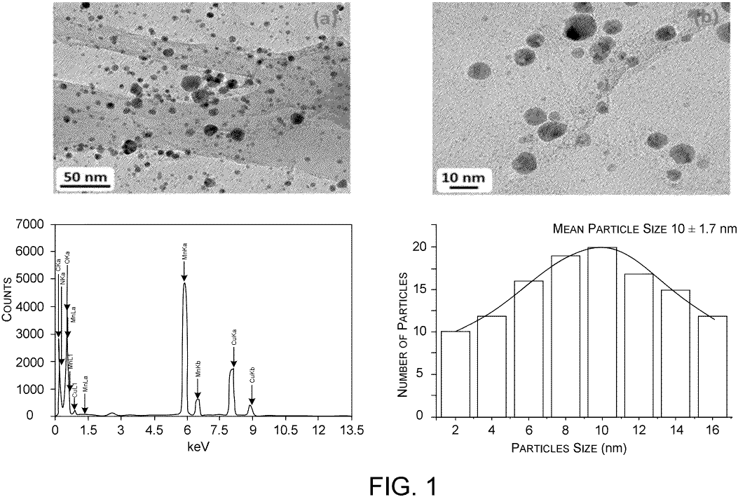

The results of high resolution transmission electron microscopy (HRTEM) displayed the existence of spherical shaped Mn₃O₄ nanoparticles on the surface of NDG within the range of 5-15 nm (FIG. 1). The Mn₃O₄ NPs are well distributed on the surface of NDG as the magnified image indicates the shape and crystallinity of these NPs. The Mn₃O₄ NPs are not bonded covalently but are held by physisorption on the NDG surface by Vander Waals interactions. The elemental composition of NDG-Mn₃O₄ nanocomposite, analyzed by energy-dispersive X-ray spectroscopy, showed intense signals at 0.65, 5.88, and 6.65 keV strongly suggesting that ‘Mn’ was the major element, which has an optical absorption in this range owing to the surface plasmon resonance (SPR). Other signals that were found in the range of 0.0-0.5 keV signified the absorption of carbon, nitrogen and oxygen, confirming the formation of NDG-Mn₃O₄ nanocomposite. The average particle size of the NDG-Mn₃O₄ nanocomposite was found to be 10 ± 1.7 nm (FIG. 1).

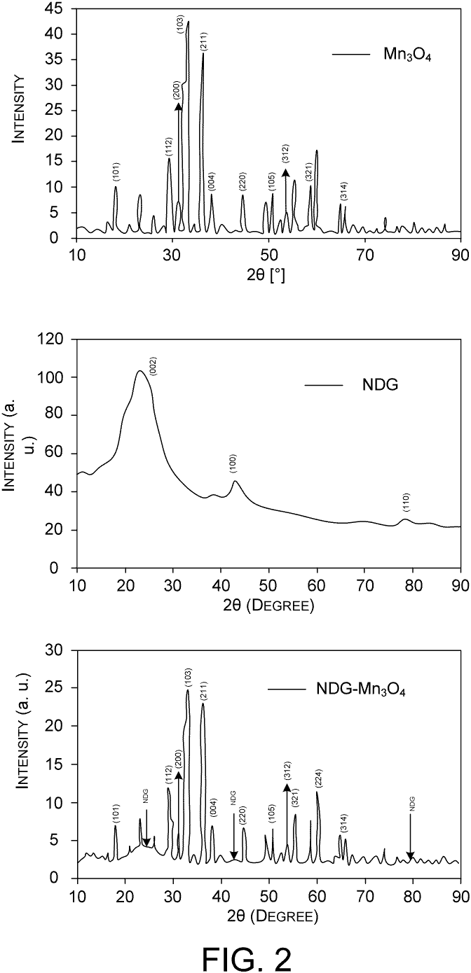

The XRD pattern of Mn₃O₄NPs shown in FIG. 2a exhibits characteristics peaks at 18.2 (101), 29.1 (112), 31.2 (200), 32.5 (103), 36.3 (211), 38.2 (004), 44.6 (220), 50.8 (105), 53.8 (312), 58.7 (321), 60.0 (224), and 64.8 (314), which points to the formation of manganese oxide NPs (FIG. 2a) These peaks reveal that the as-obtained Mn₃O₄NPs exist in single phase hexagonal wurtzite structure, besides, the data clearly matched with the standard Mn₃O₄ phase reported in the literature (JCPDS Card No. 24-0734). Notably, the sharp diffraction peaks point toward the highly crystalline and well-disperse nature of nanoparticles which clearly matched with the Hausmannite crystal phase. On the other hand, the XRD pattern of NDG-Mn₃O₄ nanocomposite showed the appearance of a broad peak at ~22.4 (002) (FIG. 2b) that confirmed the reduction of graphene oxide and formation of NDG. Furthermore, there is no broadening or shift of the (002) peak, proving that there is no change in the interlayer spacing of graphene after nitrogen-doping. No significant change in the full width at half-maximum (FWHM) of the (002) diffraction peak indicates the similar crystallite size before and after nitrogen doping. In case of the composite, the XRD pattern of which is shown in FIG. 2c, characteristic diffraction peaks of both Mn₃O₄ and N-doped graphene are present, which clearly indicate the formation of hybrid material.

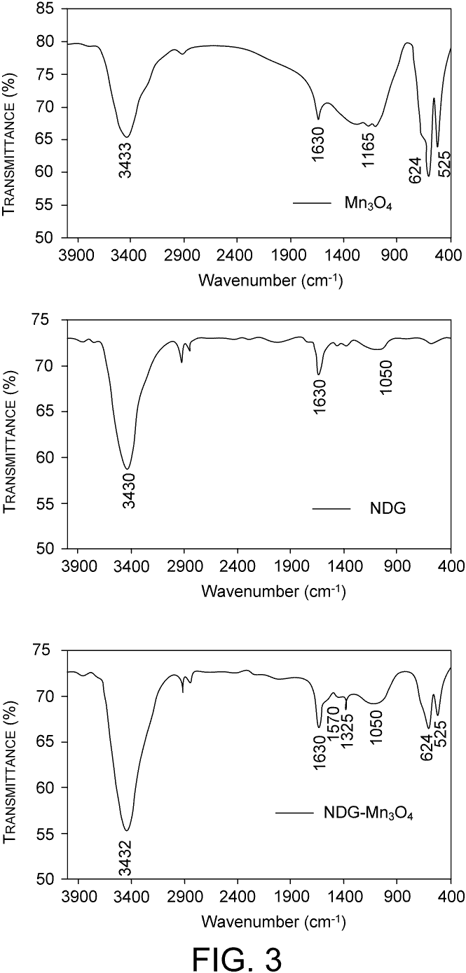

FT-IR spectra of Mn₃O₄ NPs displayed the characteristic peak of Mn-O, stretching mode in the range of 624 cm⁻¹ while the vibrational frequency associated to the Mn-O distortion vibration poisoned at 525 cm⁻¹ (FIG. 3a). The characteristic narrow and broad bands located at 3420 and 1600 cm⁻¹ were related to the hydroxyl (—OH) groups absorbed by the samples or potassium bromide. FT-IR spectra of NDG are shown in FIG. 3b. FT-IR spectra of NDG-Mn₃O₄ displayed the graphene oxide intense bands for C=C stretching (~1630 cm⁻¹), C—O—C stretching (~1209 cm⁻¹), C—O stretching (~1050 cm⁻¹). The nitrogen doping in the sample was confirmed by the presence of two characteristic peaks at ~1325 and ~1570 cm⁻¹, which were attributed to the stretching of the C—N bond from the secondary aromatic amine, which pointed toward bonding between carbon and nitrogen including the existence of other absorption bands of ‘Mn’ at 624 and 525 cm⁻¹ clearly indicating the formation of HRG—Mn₃O₄ nanocomposite (FIG. 3c).

### Cell Viability Analysis

The 3-(4,5-dimethylthiazol-2-yl)-2,5-diphenyltetrazolium bromide (MTT) method was used for testing the cytotoxicity of Mn₃O₄ and NDG—Mn₃O₄ nanoparticles. A549 lung cancer cells were seeded into 96-well plate (4 × 10⁴ cells per well) in RPMI and incubated at 37° C. for 4 h in a 5% CO₂ incubator. Different concentrations (6.25-100 µg/mL) of Mn₃O₄ and NDG—Mn₃O₄ nanoparticles were added to the 96-well plate. Phosphate buffer saline (PBS) was used as a control whereas triton-X100 was used as negative control. The cells were treated with a 670 nm laser irradiation at 0.1 W/cm² for 5 min and further incubated for 24 h. Aqueous solution of MTT (50 µL) was added to each well in the 96-well plate 4 h before the termination of 24 h incubation. The upper layer of the solution was discarded. The MTT solubilization solution, DMSO (100 µL) was added to each well to dissolve the formazan crystals by pipette stirring and then observed the absorbance at 590 nm, which was converted to cell viability using the following equation. Cell viability (%) = (absorbance of sample cells/absorbance of control cells) × 100

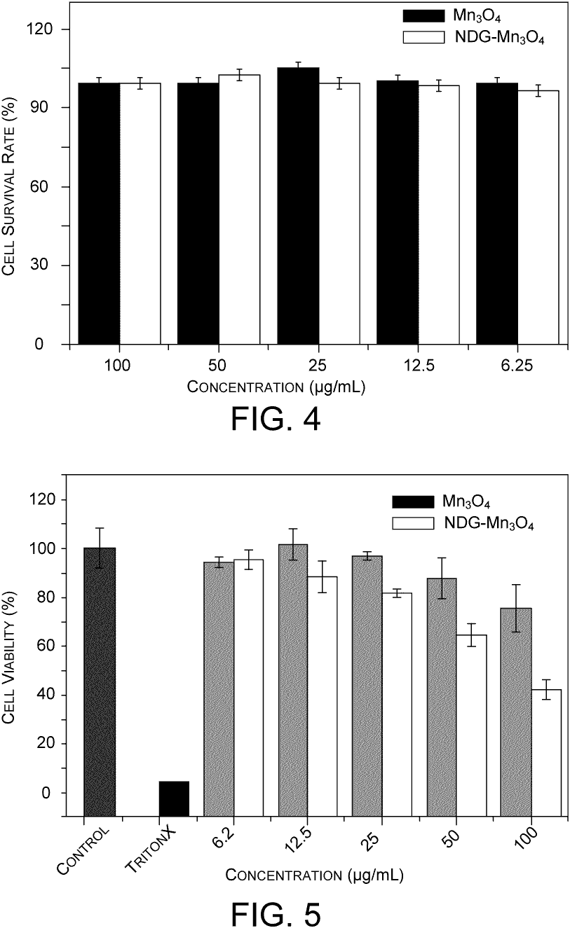

The results of cell viability analysis using MTT assay showed that exposure of Mn₃O₄ and NDG-Mn₃O₄ in the concentration range of 6.25-100 µg/mL did not cause any cytotoxicity (FIG. 4). However, NDG-Mn₃O₄ nanocomposites displayed significant cells death under laser irradiation for 5 min, while PBS (control) and Mn3O4 NPs showed negligible cell death (FIG. 5). Almost 100% cells were viable when treated with PBS whereas 41% for cancer cells survived after the treatment of 100 µg/mL concentration of NDG-Mn₃O₄ nanocomposites along with 5 min of laser irradiation. The effect of NDG-Mn₃O₄ nanocomposites on the cytotoxicity of A549 cells was concentration-dependent and only the concentrations of 25 µg/mL and above were found to be effective in killing the cells under laser irradiation (FIG. 5).

### In-Vitro Photodynamic Therapy

Fluorescence microscopy was used for morphological analysis of cancer cells following treatment with nanoparticles and laser irradiation. Fluorescein diacetate (FDA) and propidium iodide (PI) were used to visualize the live and dead cells, respectively. A549 cells (2 × 10⁴ cells per well) were seeded in a 24 well plate and incubated at 37° C. for 24 h in an atmosphere of 5% CO₂. Mn₃O₄ and NDG-Mn₃O₄ nanoparticles (50 µg/mL) were added to the wells and the plate was incubated for 4 h. After incubation, the cells were irradiated for 5 min with a 670 nm laser, followed by another incubation for 24 h. Both the dyes were added to wells and the plate was incubated for 5 min. Then, the cells were washed three times with PBS to remove excess dyes, and the fluorescence images were acquired by fluorescence microscope with 490 nm excitation and 525 nm emission wavelengths.

The results of in-vitro photodynamic therapy are shown in FIG. 6. Without laser irradiation, none of the treatments including PBS, Mn₃O₄, or NDG-Mn₃O₄ caused any cellular damage as almost all the cells appeared green. After 5 min laser irradiation, NDG-Mn₃O₄ nanocomposites killed 68% of the cancer cells (shown as red dots) whereas the treatments of PBS and Mn₃O₄ did not cause any significant cellular damage under laser irradiation (FIG. 6).

### Analysis of Singlet Oxygen Generation

1,3-Diphenylisobenzofuran (DBPF) was used to detect singlet oxygen (¹O₂) generation by NDG-Mn₃O₄ nanocomposites under 670 nm laser irradiation (0.1 W/cm²). Fifty microliters of ethanolic solution of DPBF (1 mg/mL) were added to the nanocomposites solution under stirring and irradiated with laser for different time points. The absorbance of solution was measured by UV-Visible spectrophotometer. The decrease in absorbance at 426 nm indicated the degradation of DPBF in presence of ¹O₂ which was generated by laser-induced activation of NDG-Mn₃O₄ nanocomposites.

To evaluate the ¹O₂ generation from NDG-Mn₃O₄ nanocomposites under laser irradiation, we measured the absorbance of 1,3-diphenylisobenzofuran (DPBF) after laser irradiation (670 nm, 0.1 W/cm²) at different time points (FIG. 7). The DPBF absorbance decreased with increasing the laser irradiation time, indicating the generation of singlet oxygen from NDG-Mn₃O₄ nanocomposites is directly proportional to the duration of laser irradiation (FIG. 7).

### MRI Relaxivity Analysis

A series of aqueous suspensions of NDG-Mn₃O₄ nanoparticles (with Mn concentration from 0 to 1 mM) were prepared and imaged in 0.2 mL Eppendorf tubes using a 3T clinical MRI instrument (GE Signa Excite Twin-Speed, GE Healthcare, Milwaukee, WI, USA). The specific relaxivity (r₁) was calculated from linear curve generated from concentration of NDG-Mn₃O₄ nanocomposites versus 1/T₁ (s⁻¹).

For testing the effectiveness of NDG-Mn₃O₄ nanocomposites toward diagnostic standpoint, we investigated whether these nanoparticles have MRI contrast properties or not. Various concentrations of nanoparticles were subjected to imaging by 3T MRI scanner. The result demonstrated a concentration dependent enhancement of signal intensity with increasing concentration of NDG-Mn₃O₄ nanocomposites. The r1 value was found to be 0.09 mM⁻¹s⁻¹ (FIG. 8).

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

### ADVANTAGES OF THE INVENTION

The present invention provides a nanocomposite comprising nitrogen doped graphene oxide conjugated with Mn₃O₄ nanoparticle for the effective treatment of cancer using photodynamic therapy.

The present disclosure provides a nanocomposite comprising nitrogen doped graphene oxide conjugated with Mn₃O₄ nanoparticle for the magnetic resonance imaging (MRI).

1. A nanocomposite comprising a nitrogen doped graphene oxide conjugated with Mn₃O₄ nanoparticle.

2. The nanocomposite as claimed in claim 1, wherein particle size of the nanocomposite is in a range of 5 nm to 15 nm.

3. The nanocomposite as claimed in claim 1, wherein particle size of the nanocomposite is 10±1.7 nm.

4. The nanocomposite as claimed in claim 1, wherein the nitrogen doped graphene oxide and the Mn₃O₄ nanoparticle is present in the nanocomposite in a ratio of 1:1.

5. The nanocomposite as claimed in claim 1, wherein the nanocomposite is obtained using milling process.

6. A process of preparation of a nanocomposite as claimed in claim 1, wherein the process comprises the steps of:
(a) reacting manganese (II) acetylacetonate with oleylamine at a temperature in a range of 150° C. to 170° C. for a period of 8 to 12 hours to obtain Mn₃O₄ nanoparticles;
(b) reacting a suspension of graphene oxide (GO) with hydrazine hydrate in presence of a base at a temperature range of 85° C. to 95° C. for a period of 1 to 4 hours to obtain nitrogen doped graphene oxide;
(c) milling the nitrogen doped graphene oxide and the Mn₃O₄ nanoparticles for a time period in a range of 14 hours to 17 hours to obtain a nanocomposite comprising the nitrogen doped graphene oxide conjugated with the Mn₃O₄ nanoparticle.

7. The process as claimed in claim 6, wherein manganese (II) acetylacetonate and oleylamine is present in a molar ratio of 1:25.

8. The process as claimed in claim 6, wherein the base is ammonium hydroxide or potassium hydroxide.

9. The process as claimed in claim 6, wherein the nitrogen doped graphene oxide and the Mn₃O₄ nanoparticles is in 1:1 ratio.

10. The process as claimed in claim 6, wherein the temperature in step (a) is 160° C. and in step (b) is 90° C.

11. A method of treating a cancer or imaging targeted tissue in a subject, comprising:
(a) administering to the subject in need thereof an effective amount of a nanocomposite comprising nitrogen doped graphene oxide and the Mn₃O₄ nanoparticles; and
(b) exposing cancer cell or tissue of the subject to laser irradiation in presence of fluorescein diacetate (FDA) and propidium iodide (PI) for a sufficient amount of time to obtain a desired response.

12. The method as claimed in claim 11, wherein the cancer is breast cancer, prostate cancer, brain cancer, colorectal cancer, pancreatic cancer, ovarian cancer, lung cancer, cervical cancer, liver cancer, head/neck/throat cancer, skin cancer, bladder cancer and a hematologic cancer.

13. The method as claimed in claim 11, wherein the cancer is lung cancer.

14. The method cancer as claimed in claim 11, wherein the imaging is Magnetic Resonance Imaging (MRI).
